(19) United States
(12) Patent Application Publication
(10) Pub. No.: US 2023/0250456 A1
(43) Pub. Date: Aug. 10, 2023

(54) METHOD FOR PREPARATION OF 2,6-BIS(HYDROXYMETHYL)PYRIDINE VIA ENZYMATIC CATALYSIS

(71) Applicant: VIO CHEMICALS AG, Zurich (CH)

(72) Inventors: Sven PANKE, Basel (CH); Martin HELD, Basel (CH); Tsvetan KARDASHLIEV, Basel (CH)

(21) Appl. No.: 18/014,674

(22) PCT Filed: Jul. 7, 2021
(86) PCT No.: PCT/EP2021/068920

§ 371 Date: Jan. 5, 2023

(30) Foreign Application Priority Data
Jul. 7, 2020 (WO) PCT/EP2020/025321

(51) Int. Cl. C12P 17/12; C07D 213/30; C12N 9/02; C12N 15/70; C12N 1/20; C12N 9/04

(52) U.S. Cl. CPC C12P 17/12; C07D 213/30; C12N 9/0077; C12N 9/0093; C12N 15/70; C12N 1/20; C12N 9/0006; C12Y 101/01021; C12Y 207/01017; C12Y 101/01001; C12Y 117/01

(57) ABSTRACT

The present invention relates to the provision of an enzymatic method for the preparation of 2,6-bis(hydroxymethyl) pyridine (Formula I) using as substrate 2,6-Dimethlypyridine (2,6-lutidene) and the multicomponent xylene monooxygenase comprising XylM and XylA from Pseudomonas putida (Arthrobacter siderocapsulatus). The enzymatic method of the present invention is advantageous over conventional synthetic preparations, providing access to the title compound with a one-step enzymatic procedure.

### TECHNICAL FIELD OF THE INVENTION

The present invention relates to the provision of an enzymatic method for the preparation of 2,6-bis(hydroxymethyl)pyridine (Formula I).

### BACKGROUND OF THE INVENTION

2,6-bis(hydroxymethyl)pyridine (Formula I) is a compound which can serve as a versatile intermediate in the preparation of other complex products. The hydroxyl group can be converted to many other functional groups such as aldehyde groups, halogenated hydrocarbonds, amino groups etc., which are then used in the preparation of further useful compounds. Furthermore, owing to the substitution on positions 2 and 6, 2,6-bis(hydroxymethyl)pyridine can also be used in the synthesis of macrocyclic compounds. Such an example is pyclen, an azamacrocyclic framework, which incorporates an aromatic pyridine moiety to the 12-membered macrocyclic unit.

Compound of formula I can be synthesized from 2,6-lutidine II, which is an easily accessible starting material, by oxidation with KMnO₄ toward the respective dicarboxylic acid, conversion to the respective ester and finally reduction of the ester groups to alcohols (Journal of Dispersion Science and Technology 2006, 27, p. 15-21). The cited reference is silent with respect to the yield of this three-step conversion. Additionally, this synthetic approach is tedious, as it requires three overall steps and several intermediate isolations accompanied by purifications.

CN105646334A disclosed the above synthetic approach by eliminating the ester conversion step, i.e. the dicarboxylic acid is first isolated and the directly converted to the bis-alcohol. The Chinese patent application reports a combined yield of 64% for this two-step process, which is a moderate yield for such a short synthesis.

Egorov et al reported in 1985 (Prikladnaya Biokhimiya i Mikrobiologiya, 21(3), pp. 349-353) that suspensions of certain non-multiplying cells were found able to hydroxylate 2,6-dimethylpyridine to 2-methyl-6-hydroxymethylpyridine. A small quantity of 2,6-bis(hydroxymethyl)pyridine was found to be formed only by the species Sporotrichum sulfurescens ATCC 7159. It was suggested that the polarity of the substrate is increased by insertion of the first hydroxyl group, which hinders the oxidation of the second methyl group. The document disclosed that the yield could not be substantially increased by increasing the duration of the transformation reaction.

It would be desirable to develop a selective method to produce 2,6-bis(hydroxymethyl)pyridine (Formula I) from 2,6-lutidine (Formula II) without the need to isolate intermediates and with high yield, which is cost-effective from the prospect of industrial scale.

### SUMMARY OF THE INVENTION

The present invention discloses an enzymatic method for the preparation of compound of formula I, starting from 2,6-lutidine (compound of Formula II). The method disclosed herein comprises of one step, said step comprising the presence of an enzyme, which can perform the double oxidation in a selective manner.

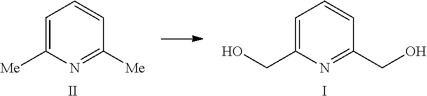

### Definitions

The following terms shall have, for the purposes of this application, including the claims appended hereto, the respective meanings set forth below. It should be understood that when reference herein is made to a general term, such as enzyme, solvent, etc. one skilled in the field may make appropriate selections for such reagents from those given in the definitions below, as well as from additional reagents recited in the specification that follows, or from those found in literature references in the field.

The term “enzymatic process” or “enzymatic method” as used herein denotes a process or method employing an enzyme or microorganism.

The term “microbial cell” refers to wild type microbial cell, wild type mutant microbial cell or genetically modified unicellular microorganism, also called recombinant, that serves as a host for production of functional entities (enzymes) participating in the enzymatic process. The terms host and cell are used interchangeably throughout the present invention.

The term “recombinant cell” denotes that the microbial cell further harbors heterologous DNA encoding enzyme functionality supplied in the form of genomic integration or plasmid DNA.

The term “feeding rate” denotes the quantity of substance (e.g. glucose or lutidine) per unit of time added to the reaction medium within the course of the enzymatic process.

The term “reaction medium” refers to any growth medium used to perform a process which comprises enzymes. Said medium is able to carry the starting material, the enzyme either alone or as part of a cell and the product and byproducts. Usually, the reaction medium is a solvent.

The term “cofactor regeneration system” denotes an enzyme or a set of enzymes that reduce a biological cofactor, preferably NAD+ to NADH, NADP+ to NADPH, GDP+ to GDPH, and more preferably of NAD+ to NADH using biocompatible substrates such as glucose, an alcohol or formate.

The term “formate” refers to the anion generated by the respective salts, e.g. sodium formate.

The enzymes employed in the present invention are derived from bacterial or fungal genomes. The genes may be codon optimized and synthetically prepared or cloned from the respective host (e.g. by PCR). For example, they may be cloned in suitable expression vectors or integrated on the genome of the recombinant host to yield genetically engineered host cells.

Additionally, it should be understood in the methods of preparation and claims herein, that the pronoun “a”, when used to refer to a reagent, such as “a base”, “a solvent” and so forth, is intended to mean “at least one” and thus, include, where suitable, single reagents as well as mixtures of reagents.

### DETAILED DESCRIPTION OF THE INVENTION

The present invention discloses an enzymatic method for the preparation of compound of 2,6-bis(hydroxymethyl)pyridine (Formula I).

The inventors have surprisingly found that it is possible to obtain compound of formula I starting from readily available 2,6-lutidine II in the presence of enzymes in high yields and without formation of significant amounts of byproducts.

It is an object of the present invention to provide a process for the transformation of 2,6-lutidine II to 2,6-bis(hydroxymethyl)pyridine I, wherein the transformation is performed in the presence of enzymes.

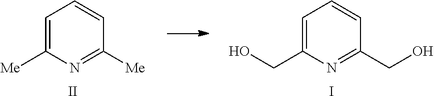

Said process comprises the step of contacting a compound of formula II with an enzyme to form compound of formula I.

In a preferred embodiment, the transformation proceeds via the formation of 6-methyl-2-hydroxypyridine III.

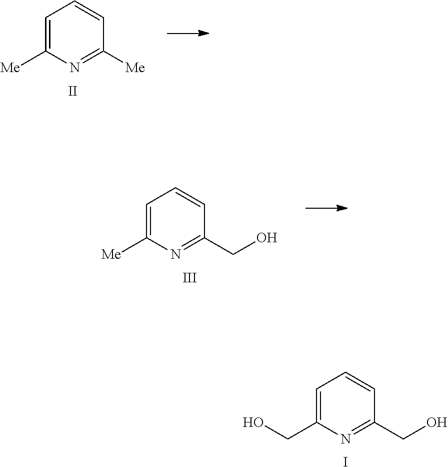

The enzyme may be one which can catalyze the oxidative transformation of the methyl groups of 2,6-lutidine to the respective hydroxymethyl groups of 2,6-bis(hydroxymethyl)pyridine I.

Preferably, the enzyme is an oxidoreductase. More preferably, the enzyme is NADH-dependent, GDPH-dependent or NADPH-dependent. Even more preferably, the enzyme is NADH-dependent.

In a preferred embodiment the oxidoreductase uses molecular oxygen to oxidize 2,6-lutidine II.

In another preferred embodiment, the oxidoreductase enzyme is capable of regioselectively oxidizing methyl groups on aromatics. More preferably, the oxidoreductase enzyme is a xylene monooxygenase enzyme encoded by the is xylM and xylA genes of Pseudomonas putida (Arthrobacter siderocapsulatus), or a XylMA-like enzyme of Alteromonas Macleodii or of Tepidiphilus Succinatimandens or of Novosphingobium Kunmingense or of Hyphomonas Oceanitis or of Sphingobium sp. 32-64-5 or of Halioxenophilus Aromaticivorans or a XylM-like enzyme with more than 70% sequence identity on the amino acid level. Even more preferably, the oxidoreductase enzyme is a xylene monooxygenase enzyme encoded by the xylM and xylA genes of Pseudomonas putida (Arthrobacter siderocapsulatus).

Sources of enzymes suitable for use in the present invention may be publically available (meta)genomic databases. Alternatively, the enzyme may be the result of genetic manipulation of a known enzyme.

The enzyme may be used in the disclosed method according to techniques well known to the skilled person. They may be used as part of the cells producing them (whole cell catalysis) or in vitro, where the enzyme is available and is employed in the reaction media under appropriate reaction conditions.

In a preferred embodiment, the enzyme is expressed in a microbial host. The microbial host may then be referred to as a recombinant microbial host. The recombinant host may further be tailored by genetic engineering. Preferable microbial hosts are Escherichia coli, Corynebacterium glutamicum, Bacillus subtilis, Pseudomonas putida, Rhodobacter sphaeroides, Streptomyces spp, Propionibacterium shermanii, Ketogulonigenium vulgare, Acinetobacter baylyi, Halomonas bluephagenesis. More preferable is Escherichia coli.

The skilled person is familiar with techniques for expressing certain enzymes in microbial hosts. Such techniques are exemplified in relevant textbooks, such as “Methods in Enzymology” (Book series, Elsevier, ISSN 0076-6879) or “Molecular Cloning” (ISBN 978-1-936113-42-2).

The enzymatic process disclosed herein preferably proceeds via the formation of 6-methyl-2-hydroxypyridine III.

The inventors have found that, in addition to compound of formula III, the enzymatic transformation of compound of formula II to compound of formula I proceeds via the formation of compound of formula IV, when the enzyme is a xylene monooxygenase enzyme.

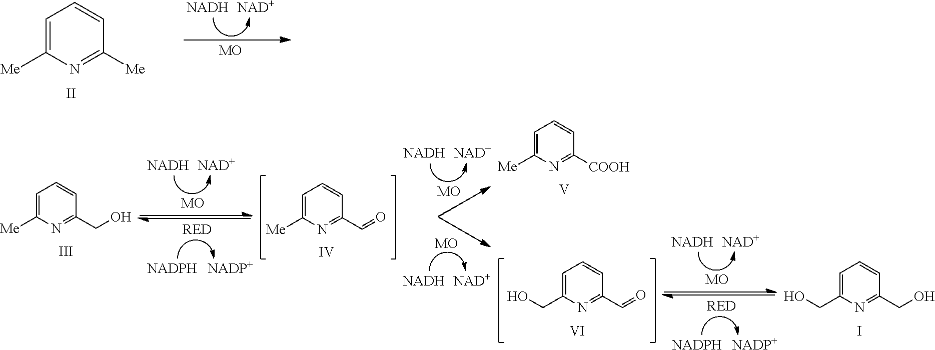

It is therefore important that compound of formula II is kept at a feeding rate suitable for maintain a balance between the various transformations occurring within the enzymatic process. The feeding rate need not be constant, as long as it is adjusted according to the below embodiments. The feeding rate should also be at an appropriate level so as not to reach growth-inhibitory levels. 2,6-lutidine II concentrations exceeding 1 g/L become growth-inhibitory.

In a preferred embodiment, the feeding rate of 2,6-lutidine II in the reaction medium is adjusted such that the concentration of 2,6-lutidine II does not exceed the value of 1 g/L, preferably 0.1 g/L, and more preferably 0.02 g/L in the reaction medium.

In another preferred embodiment, the feeding rate of 2,6-lutidine II in the reaction medium is adjusted such that the concentration of 2,6-lutidine II does not fall below the value of 10 mg/L, preferably 0.1 mg/L, more preferably 0.01 mg/L.

In a more preferred embodiment, the feeding rate of 2,6-lutidine II in the reaction medium is adjusted such that the concentration of 2,6-lutidine II does not exceed the value of 1 g/L and does not fall below the value of 10 mg/L, preferably 0.1 mg/L, more preferably 0.01 mg/L.

In another preferred embodiment, the feeding rate of 2,6-lutidine II in the reaction medium is adjusted such that the concentration of 2,6-lutidine II does not exceed the value of 0.1 g/L and does not fall below the value of 10 mg/L, preferably 0.1 mg/L, more preferably 0.01 mg/L.

In another preferred embodiment, the feeding rate of 2,6-lutidine II in the reaction medium is adjusted such that the concentration of 2,6-lutidine II does not exceed the value of 0.02 g/L and does not fall below the value of 10 mg/L, preferably 0.1 mg/L, more preferably 0.01 mg/L.

The method of the present invention is conducted in an aqueous medium. The aqueous medium is water, or deionized water, which may further comprise a buffer agent.

The weight of biomass employed in the present process may be adjusted according to the skilled person's general knowledge.

The reaction medium temperature may be such that the enzyme retains its enzymatic activity. It may be adjusted according to the restrictions of the enzyme. It is preferably maintained between 25 and 37° C. preferably between 28 and 35° C.

The pH may be such that the enzyme retains its enzymatic activity. It may be adjusted according to the restrictions of the enzyme. Preferably, the pH is between 6.0 and 8.0, more preferably 6.5-7.5 and even more preferably 7.0±0.1.

The dissolved oxygen tension (DOT) should be maintained above 0%. DOT drops as cells grow and biomass accumulates in the bioreactor and furthermore there is a significant drop once the substrate is added. It is therefore important that it is maintained above 0% or better above 3-5% in order for the biocatalytic reaction to take place. DOT can be controlled by mixing speed and by air supply.

The rate of glucose feed may be adjusted as per skilled person's general knowledge.

The reaction time can be varied depending upon the amount of enzyme and its specific activity. It may further be adjusted by the temperature or other conditions of the enzymatic reactions, which the skilled person is familiar with. Typical reaction times are ranging between 1 hour and 72 hours.

In another embodiment, there is provided a process for the transformation of 2,6-lutidine II to 2,6-bis(hydroxymethyl)pyridine I, wherein the transformation is performed in the presence of enzymes, which catalyze the oxidative transformation of the methyl groups of 2,6-lutidine II to the respective hydroxymethyl groups of 2,6-bis(hydroxymethyl)pyridine I, and, additionally the presence of a dehydrogenase.

The transformation may be performed directly in the microbial cell with no further engineering of the housekeeping dehydrogenases.

In yet another embodiment, the microbial cell further synthesizes a dehydrogenase from another microbial cell.

In even yet another embodiment, one or more housekeeping dehydrogenases are deactivated or engineered.

In a preferred embodiment, the microbial cell further synthesizes a dehydrogenase from another microbial cell and one or more housekeeping dehydrogenases are deactivated or engineered.

The enzyme which catalyzes the oxidative transformation of the methyl groups of 2,6-lutidine II to the respective hydroxymethyl groups of 2,6-bis(hydroxymethyl)pyridine I and is employed in this embodiment is according to the previous embodiments.

As long as the skilled person arrives at a specific combination of enzymes, the expression of them within the same microbial host is a technique well known to the skilled person. Reference books have been provided above.

In a preferred embodiment, the dehydrogenase is NAD(P)H dependent or NADH dependent and preferentially NADH dependent.

In another preferred embodiment, the dehydrogenase catalyzes the reduction of 6-methylpyridine-2-carboxaldehyde IV to 6-methyl-2-hydroxypyridine III or the reduction of 6-(hydroxymethyl)-2-pyridinecarbaldehyde V to 2,6-bis(hydroxymethyl)pyridine I. Preferably, the dehydrogenase catalyzes both the reduction of 6-methylpyridine-2-carboxaldehyde IV to 6-methyl-2-hydroxypyridine III and the reduction of 6-(hydroxymethyl)-2-pyridinecarbaldehyde V to 2,6-bis(hydroxymethyl)pyridine I.

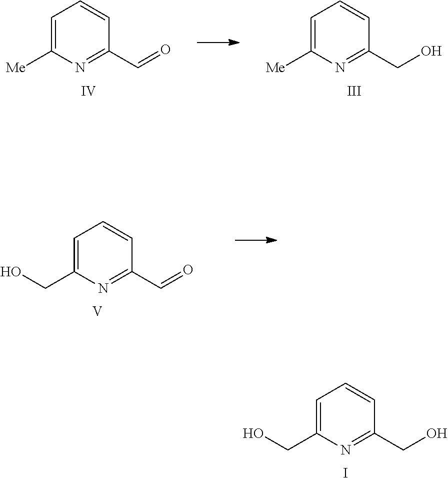

In another preferred embodiment, the dehydrogenase is selected from the list of the AKR from Kluyveromyces lactis, XylB from Acinetobacter baylyi ADP1, and AFPDH from Candida maris.

In another embodiment, there is provided a process for the transformation of 2,6-lutidine II to 2,6-bis(hydroxymethyl)pyridine I, wherein the transformation is performed in the presence of enzymes, which catalyze the oxidative transformation of the methyl groups of 2,6-lutidine II to the respective hydroxymethyl groups of 2,6-bis(hydroxymethyl)pyridine I, and additionally the presence of a co-factor regeneration system.

The enzyme, which catalyzes the oxidative transformation of the methyl groups of 2,6-lutidine II to the respective hydroxymethyl groups of 2,6-bis(hydroxymethyl)pyridine I and is employed in this embodiment, is according to the previous embodiments.

The transformation may be performed directly in the microbial cell with no further engineering of the housekeeping dehydrogenases, as disclosed in previous embodiments.

In yet another embodiment, the microbial cell further synthesizes a dehydrogenase from another microbial cell.

In even yet another embodiment, one or more housekeeping dehydrognases are deactivated or engineered.

In a preferred embodiment, the microbial cell further synthesizes a dehydrogenase from another microbial cell and one or more housekeeping dehydrogenases are deactivated or engineered.

The dehydrogenase employed in this embodiment is according to the previous embodiments.

The co-factor may be NAD(P)H or NADH and the regeneration system is a NAD(P)H or NADH regeneration system. Preferably, the regeneration system is a NADH regeneration system.

The regeneration system is preferably co-expressed in the same microbial host which expresses the enzyme catalyzing the oxidative transformation. In a more preferred embodiment, the same microbial host co-expresses also a dehydrogenase, as described in previous embodiments.

Cofactors are non-protein chemical compounds that play an essential role in many enzyme catalysed biochemical reactions. Cofactors act to transfer chemical groups between enzymes. Nicotinamide adenine dinucleotide (NAD+), and nicotinamide adenine dinucleotide phosphate (NADP+) and the reduced forms of said molecules (NADH and NADPH, respectively) are biological cofactors which play a central role in the metabolism of cells acting as electron transfer agents. The oxidized forms NAD+ and NADP+ act as electron acceptors, becoming reduced in the process. NADH and NADPH, in turn, can act as reducing agents, becoming oxidized in the process. Most enzymes that mediate oxidation or reduction reactions are dependent on cofactors such as NADPH or NADH. Cofactor regeneration systems are employed to ensure that the cofactor participating within a given bioprocess is not depleted and/or to reduce the total cost of the process.

In a preferred embodiment, the NADH regeneration system is a formate dehydrogenase regeneration system.

In another preferred embodiment, the NADH regeneration system is a formate dehydrogenase-based system, more preferably a cytosolic format dehydrogenase with no sensitivity towards oxygen.

In another preferred embodiment, the NADH recycling system is comprised of a metal-independent formate dehydrogenase active on NAD+ species and of bacterial or fungal origin.

Preferably, the metal-independent formate dehydrogenase, which is active on NAD+ species, is from Candida tropicalis or Mycobacterium vaccae FDH.

In a preferred embodiment, the formate is fed to the process, as defined in any of the previous embodiments, for regeneration of NADH consumed by the enzyme, which catalyzes the oxidative transformation of the methyl groups of 2,6-lutidine II to the respective hydroxymethyl groups of 2,6-bis(hydroxymethyl)pyridine I, the dehydrogenase, or both. Preferably, the formate is fed to the process, for regeneration of NADH consumed by the oxidoreductase, the dehydrogenase, or both.

In a preferred embodiment, the feeding rate of formate in the reaction medium is adjusted such that the concentration of formate does not exceed the value of 150 mM, preferably 100 mM, more preferably 50 mM.

In another preferred embodiment, the feeding rate of formate in the reaction medium is adjusted such that the concentration of formate does not fall below the value of 50 mM, preferably 25 mM, more preferably 5 mM, in the reaction medium.

In a more preferred embodiment, the feeding rate of formate in the reaction medium is adjusted such that the concentration of 2,6-lutidine II does not exceed the value of 150 mM and does not fall below the value of 50 mM, preferably 25 mM, more preferably 5 mM, in the reaction medium.

In another preferred embodiment, the feeding rate of formate in the reaction medium is adjusted such that the concentration of formate does not exceed the value of 100 mM and does not fall below the value of 50 mM, preferably 25 mM, more preferably 5 mM, in the reaction medium.

In another preferred embodiment, the feeding rate of formate in the reaction medium is adjusted such that the concentration of formate does not exceed the value of 50 mM and does not fall below the value of 50 mM, preferably 25 mM, more preferably 5 mM, in the reaction medium.

### EXAMPLES

### Example 1: Conversion of Lutidine by Recombinant E. coli Expressing XylMA Protein in Shake Flasks

The polynucleotide sequence of the xylM and xylA genes of Pseudomonas putida (Arthrobacter siderocapsulatus) encoding for mutlicomponent xylene monooxygenase, XylMA, was cloned into plasmid (pBR322 origin of replication, kan gene encoding kanamycin resistance protein and inducible P_{alkS} promoter for XylMA induction by dicyclopropyl ketone (DCPK)) and transformed by electroporation into an E. coli BL21 host. A single colony was propagated 37° C., 200 rpm for 12-14 h in 4 mL LB growth medium. On the following day, the overnight culture in LB was used to innoculate a main culture in minimal medium containing 4.5 g/L KH₂PO₄, 6.3 g/L Na₂HPO₄, 2.3 g/L (NH₄)₂SO₄; 1.9 g/L NH₄Cl; 1 g/L citric acid, 20 mg/L thiamine, 10 g/L glucose, 55 mg/L CaCl₂), 240 mg/L MgSO₄, 1× trace elements (0.5 mg/L CaCl₂). 2H₂O; 0.18 mg/L ZnSO₄. 7H₂O, 0.1 mg/L MnSO₄. H₂O, 20.1 mg/L Na₂-EDTA, 16.7 mg/L FeCl₃. 6H₂O, 0.16 mg/L CuSO₄.5H₂O), 50 mg/L kanamycin at pH 7 with NH₄OH. The starting optical density (OD600) of the 20 mL main culture in 100 mL shake flask was adjusted to 0.05 and the flask was incubated at 37° C., 200 rpm until OD of 0.6-0.8 was reached, then 0.025% DCPK was added and the culture was further incubated at 30° C., 200 rpm for another hour or until OD reached 1. At the target OD, various sub-growth-inhibitory concentrations of 2,6 lutidine II were added to the cells and the cultures were incubated further until complete substrate conversions was achieved and cell growth has stalled for at least 2 hours. The reaction progression was monitored and quantified using RP-HPLC equipped with a C18 column at 270 nm and a specific activity range of 0.3-0.6 g/gCDW/h were calculated for the individual reactions catalyzed by the whole cells. Up to 1.25 g/L total product (90% 2,6-bis(hydroxymethyl)pyridine I; 10% 6-methyl-2-pyridinecarboxylic acid V) from 10 g/L glucose.

### Example 2: Conversion of Lutidine by Recombinant E. coli Expressing XylMA Protein in a Bioreactor

The microbial strain, media and growth conditions up to inoculation of main culture are identical to example one. In this example, the main culture is prepared in bioreactor where parameters such as temperature, pH, dissolved oxygen tension, mixing and glucose availability can be controlled allowing for fed batch fermentations. Fluctuations in pH are maintained by appropriate addition of ammonium hydroxide or sulfuric acid controlled by a pH-stat. For the batch phase of the fermentation, 1 L growth media (as in example 1) was inoculated at a starting OD600 of 0.025 and cells were grown at 30° C. for 12-13 h or until they completely consumed the initially provided carbon source (glucose) which is indicated by a sharp jump in dissolved oxygen in the bioreactor. At this stage, the fed-batch phase of the fermentation is added by initiation an appropriate glucose feed rate from a 500 g/L glucose stock supplemented with 1× trace elements, 1× kanamycin and 240 mg/L MgSO₄ such that a growth rate of 0.31 h⁻¹ was maintained until OD600 reached 35 when 0.05% DCPK were added. One hour post induction with DCPK, 2,6-Lutidine II was added to the bioreactor (feed rate: 0.1 mL/L of broth/min) and the reaction was let to proceed for 14-18 h. A second substrate addition can be made once the initial amount is fully converted to 2,6-bis(hydroxymethyl)pyridine I and the reaction is let to proceed until conversion is completed or as long growth rate of the cells higher than 0.025 h⁻¹ is maintained. Up to 15 g/L total product (90% 2,6-bis(hydroxymethyl)pyridine I; 10% 6-methyl-2-pyridinecarboxylic acid V) could be produced within 18 h biotransformation.

### Example 3: Conversion of Lutidine by E. coli Recombinantly Expressing XylMA, NADH-Dependent Aldo-Keto Reductase and Formate Dehydrogenase in a Bioreactor

The polynucleotide sequences of the xylM and xylA genes of Pseudomonas putida (Arthrobacter siderocapsulatus) encoding for mutlicomponent xylene monooxygenase, XylMA, akr gene (e.g klakr of Kluyveromyces lactis XP1461) encoding for a NADH-dependent aldo-keto reductase, and fdh gene (e.g., cbfdh is of Candida boidinii or mcfdh of Mycolicibacterium vaccae) encoding for a NADH-dependent formate dehydrogenase were genome integrated in microbial E. coli BL21 under inducible promoters P_{alkS}, P_{trc} and P_{tac}, respectively. Overnight culture from a single colony propagated at 37° C., 200 rpm for 12-14 h in LB medium. For the batch phase of the fermentation, 1 L minimal growth media (as in example 1) was inoculated at a starting OD600 of 0.025 and cells were grown at 30° C. for 12-13 h or until they completely consumed the initially provided carbon source (glucose) which is indicated by a sharp jump in dissolved oxygen in the bioreactor. The fed-batch/protein expression phase of the fermentation initiated by an appropriate glucose feed rate from a 500 g/L glucose stock supplemented with 1× trace elements, 1× kanamycin and 240 mg/L MgSO₄ such that a growth rate of 0.2 h⁻¹ was maintained until OD600 reached 30. Then, 0.025 mM IPTG were added to induce expression of the XXXX dehydrogenase XXXX and formate dehydrogenase and cells were grown at the aforementioned growth rate. When optical density (0D600) reached 60, 0.025% DCPK were added to induce the expression of XylMA. When cells reached optical density of 80 biotransformation was initiated by adding the substrate solution (50 mL) containing lutidine and formate (resulting in a final concentration of 0.2-0.4 M each) at a rate of 0.85 mL/min. Samples were collected at different time points for 12-16 h from the biotransformation initiation phase for product quantification by HPLC. Up to 20 g/L total product (90% 2,6-bis(hydroxymethyl)pyridine I; 10% 6-methyl-2-pyridinecarboxylic acid V) were produced in 24 h biotransformation.

1. A process for the transformation of 2,6-lutidine II to 2,6-bis(hydroxymethyl)pyridine I, wherein the transformation is performed in the presence of enzymes.

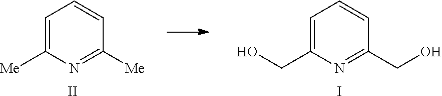

2. A process according to claim 1, wherein the transformation proceeds via the formation of 6-methyl-2-hydroxypyridine III.

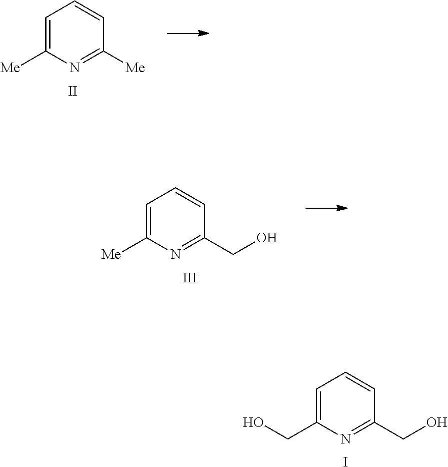

3. A process according to claims 1-2, wherein the enzymes are oxidoreductases.

4. A process according to claim 3, wherein the oxidoreductase is NADH dependent.

5. A process according to claims 3-4, wherein the oxidoreductase uses molecular oxygen to oxidize 2,6-lutidine II.

6. A process according to claims 3-5, wherein the oxidoreductase enzyme is a xylene monooxygenase enzyme encoded by the xylM and xylA genes of Pseudomonas putida (Arthrobacter siderocapsulatus), or a XylMA-like enzyme of Alteromonas Macleodii or of Tepidiphilus Succinatimandens or of Novosphingobium Kunmingense or of Hyphomonas Oceanitis or of Sphingobium sp. 32-64-5 or of Halioxenophilus Aromaticivorans or a XylMA-like enzyme with more than 70% sequence identity on the amino acid level.

7. A process according to claim 6, wherein the oxygen-incorporating enzyme is a xylene monooxygenase enzyme comprising a xylM subunit and a xylA subunit.

8. A process according to any preceding claim wherein the enzyme is expressed in a microbial host.

9. A process according to claims 6 and 7, wherein the xylM and xylA subunits are expressed in a microbial host.

10. A process according to claims 8 and 9, wherein the microbial host is Escherichia coli, Corynebacterium glutamicum, Bacillus subtilis, Pseudomonas putida, Rhodobacter sphaeroides, Streptomyces spp, is Propionibacterium shermanii, Ketogulonigenium vulgare, Acinetobacter baylyi, Halomonas bluephagenesis.

11. A process according to any preceding claim, wherein the feeding rate of 2,6-lutidine II in the reaction medium is adjusted such that the concentration of 2,6-lutidine II does not exceed the value of 1 g/L, preferably 0.1 g/L, and more preferably 0.02 g/L in a reaction medium.

12. A process according to any preceding claim, wherein the feeding rate of 2,6-lutidine II in the reaction medium is adjusted such that the concentration of 2,6-lutidine II does not fall below the value of 10 mg/L, preferably 0.1 mg/L, more preferably 0.01 mg/L.

13. A process according to claims 8-12, wherein a dehydrogenase is co-expressed in the microbial host.

14. A process according to claim 13, wherein the dehydrogenase is NADH dependent, NADP dependent, NADPH dependent or GDH dependent.

15. A process according to claims 13-14, wherein the dehydrogenase catalyzes the reduction of 6-methylpyridine-2-carboxaldehyde IV to 6-methyl-2-hydroxypyridine III and 6-(hydroxymethyl)-2-pyridinecarbaldehyde V to 2,6-bis(hydroxymethyl)pyridine I.

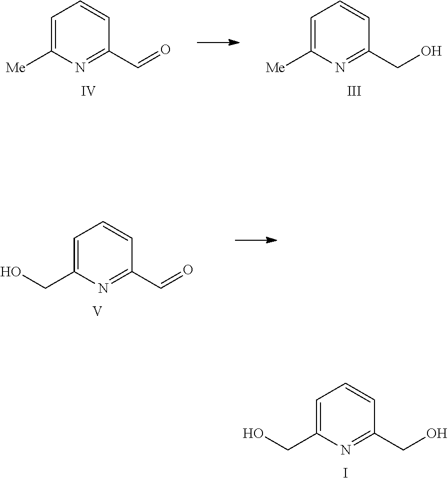

16. A process according to claims 13-15, wherein the dehydrogenase is selected from the list of the AKR from Kluyveromyces lactis, XylB from Acinetobacter baylyi ADP1, and AFPDH from Candida maris.

17. A process according to any preceding claim, wherein a NADH regeneration system, a NADP regeneration system, a NADPH regeneration system or a GDH regeneration system is co-expressed in the microbial host.

18. A process according to claim 17, wherein the NADH regeneration system is a formate dehydrogenase-based system.

19. A process according to claims 17-18, wherein the NADH regeneration system is comprised of a metal-independent formate dehydrogenase active on NAD+ species and of bacterial or fungal origin.

20. A process according to claims 17-19, wherein the NADH regeneration system is comprised of a metal-independent formate dehydrogenase active on NAD+ species from Candida tropicalis or Mycobacterium vaccae FDH.

21. A process according to claims 17-20, wherein formate is fed to the process, as defined in claim 1, 2 or 14, for regeneration of NADH consumed by the oxidoreductase, the dehydrogenase, or both.

22. A process according to claim 21, wherein the feeding rate of formate is such that the concentration of formate in the reaction medium does not exceed the value of 150 mM, preferably 100 mM, more preferably 50 mM.

23. A process according to claims 21 and 22, wherein the feeding rate of formate is such that the concentration of formate does not fall below the value of 50 mM, preferably 25 mM, more preferably 5 mM, in the reaction medium.
